(19) United States
(12) Patent Application Publication
(10) Pub. No.: US 2025/0177020 A1
(43) Pub. Date: Jun. 5, 2025

(54) PROSTHETIC DEVICE

(71) Applicant: ENCORE MEDICAL, L.P. (D/B/A DJO SURGICAL), Austin, TX (US)

(72) Inventors: Jonathan LEVY, Austin, TX (US); Jeremy MCLAIN, Austin, TX (US); Alysha ROBICHEAUX, Austin, TX (US)

(21) Appl. No.: 18/845,845

(22) PCT Filed: Oct. 4, 2023
(86) PCT No.: PCT/US2023/034493

§ 371 Date: Sep. 10, 2024

Related U.S. Application Data
(60) Provisional application No. 63/413,761, filed on Oct. 6, 2022.
(60) Provisional application No. 63/380,018, filed on Oct. 18, 2022.
(60) Provisional application No. 63/520,400, filed on Aug. 18, 2023.

(51) Int. Cl. A61B 17/84; A61F 2/30

(52) U.S. Cl. CPC A61B 17/846; A61F 2/30749; A61F 2/30767

(57) ABSTRACT

A prosthetic device includes a baseplate with a peripheral bore, a central screw extending from a bone-contacting surface of the baseplate, and a peg having an osteogenic coating. The peg is able to couple to the peripheral bore and extend a first length beyond the bone-contacting surface of the baseplate and into a hole formed within a bone. The depth of the hole in the bone is greater than the first length.

### CROSS-REFERENCE TO RELATED APPLICATIONS

This application claims the benefit of, and priority to, U.S. Provisional Patent Application No. 63/413,761 filed on Oct. 6, 2022, U.S. Provisional Patent Application No. 63/380,018 filed on Oct. 18, 2022, and U.S. Provisional Patent Application No. 63/520,400 filed on Aug. 18, 2023, each of which is hereby incorporated by reference herein in its entirety.

### TECHNICAL FIELD

The present disclosure relates generally to orthopedics and, more specifically, components to insert into bone.

### BACKGROUND

Orthopedic surgeons seek to replace or correct musculoskeletal components affected by trauma, injury, and/or disease. Joint replacement surgeries, such as for hips or shoulders, are fairly common orthopedic surgeries. Such surgeries require an implant to be inserted into bone to help secure a joint replacement component, such as a ball or socket component of a hip or shoulder joint. There are two types of bone-cortical and cancellous. Cortical bone is the hard outer layer, and cancellous bone is the spongy internal layer of the bone. Existing bone implants often use long, bi-cortical screws that extend through a first cortical bone layer, a cancellous bone layer, and finally through a second cortical bone layer. Some patients do not have enough dense bone available to provide adequate screw fixation and may have to forego the use of a screw in that region, potentially decreasing the overall construct fixation. A need exists for systems, methods, and apparatuses for prosthetic devices that can be inserted when bi-cortical screw fixation cannot be achieved, and additional peripheral enhanced fixation is beneficial.

### SUMMARY

According to some implementations of the present disclosure, a prosthetic device includes a baseplate with a peripheral bore, a central screw extending from a bone-contacting surface of the baseplate, and a peg having an osteogenic coating. The peg is able to couple to the peripheral bore and extend a first length beyond the bone-contacting surface of the baseplate and into a hole formed within a bone. The depth of the hole in the bone is greater than the first length.

According to some implementations of the present disclosure, a method of installing a prosthetic device includes providing a prosthetic device. The prosthetic device includes a baseplate, a central screw, and a peg. The baseplate includes a peripheral bore. The peg is of a first length. The method also includes providing a bone and drilling a hole of a second length into the bone. The method includes inserting the central screw through the baseplate and into the bone. The method further includes inserting the peg through the peripheral bore and into the hole.

### BRIEF DESCRIPTION OF THE DRAWINGS

Disclosed herein are implementations of systems, apparatuses, and methods pertaining to a prosthetic device. This description includes drawings, wherein:

FIG. 1A is a top perspective view of an assembled modular prosthetic device, according to some implementations;

FIG. 1B is a bottom perspective view of an assembled modular prosthetic device, according to some implementations;

FIG. 2A is a top perspective view of an exploded modular prosthetic device, according to some implementations;

FIG. 2B is a bottom perspective view of an exploded modular prosthetic device, according to some implementations;

FIGS. 3A and 3B are side and cross-sectional views of a peg, according to some implementations;

FIG. 4 is a cross-sectional view of a prosthetic device implanted in a shoulder, according to some implementations;

FIG. 5 is a method of installing a prosthetic device, according to some implementations;

FIG. 6A is a top perspective view of an assembled monolithic prosthetic device, according to some implementations;

FIG. 6B is a bottom perspective view of an assembled monolithic prosthetic device, according to some implementations;

FIG. 7A is a top perspective view of an exploded monolithic prosthetic device, according to some implementations;

FIG. 7B is a bottom perspective view of an exploded monolithic prosthetic device, according to some implementations; and

FIG. 8 is a top perspective view of an exploded monolithic prosthetic device with an implant component for use with the prosthetic device, according to some implementations.

### DETAILED DESCRIPTION

The present disclosure is described with reference to the attached figures, wherein like reference numerals are used throughout the figures to designate similar or equivalent elements. Several aspects of the present disclosure are described below with reference to example implementations for illustration.

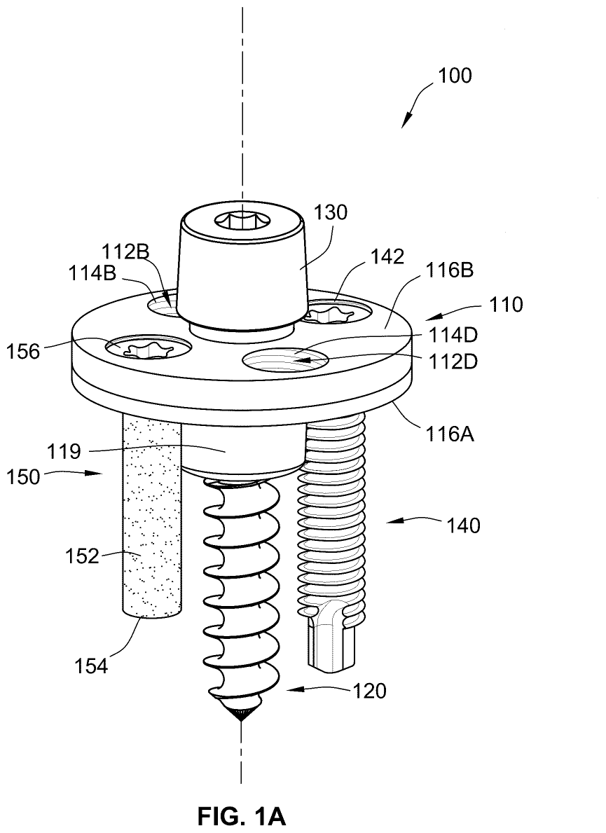

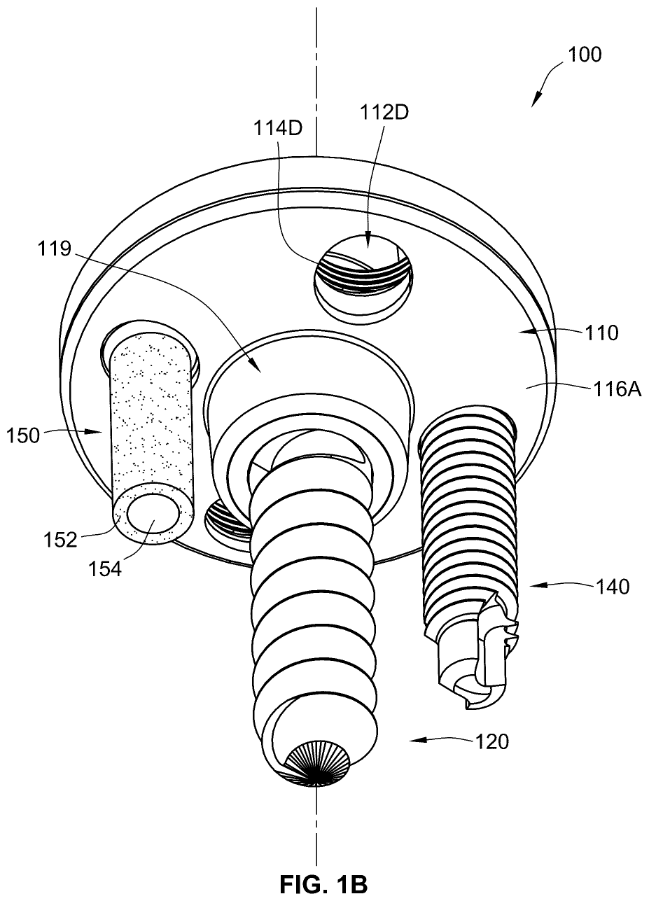

Described herein are systems, methods, and apparatuses that seek to minimize, if not eliminate, the drawbacks of the currently available bone implants. For example, in some implementations, the systems, methods, and apparatuses described herein do not use any, or use relatively fewer, bi-cortical screws (e.g., some implementations of the present disclosure use 1, 2, or 3 screws instead of 4 screws). Rather, the systems, methods, and apparatuses described herein utilize at least one peripheral peg in addition to, or in lieu of, one or more traditional bi-cortical screws. The reduced reliance on bi-cortical screws to implant the prosthetic device also allows greater access to joint replacement surgery because a relatively lesser amount of dense bone (e.g., bone having a hardness sufficient to receive and sufficiently maintain a bi-cortical screw therein) is needed. The discussion of FIGS. 1A-3B provides an overview of such prosthetic devices.

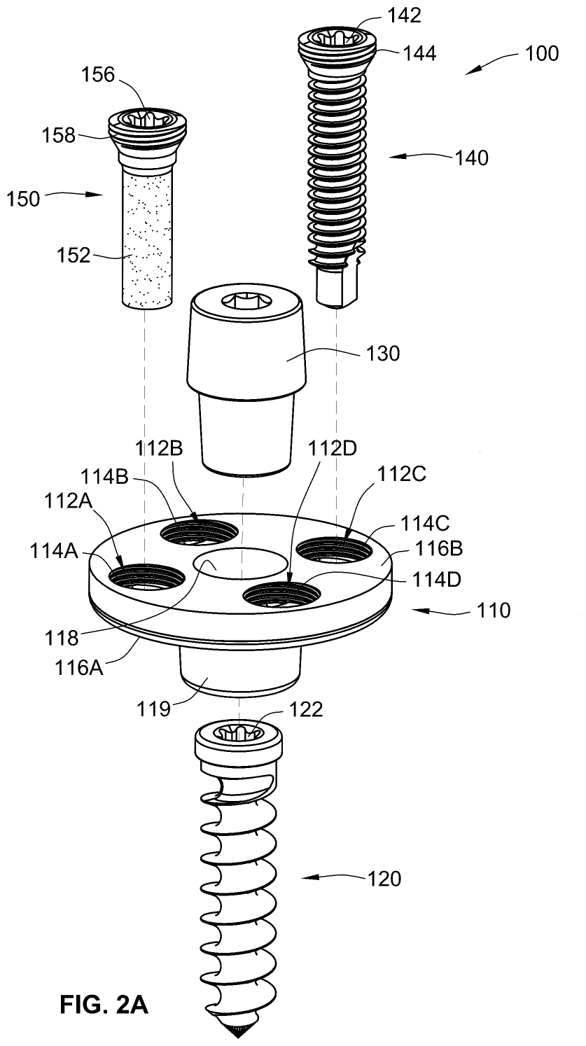

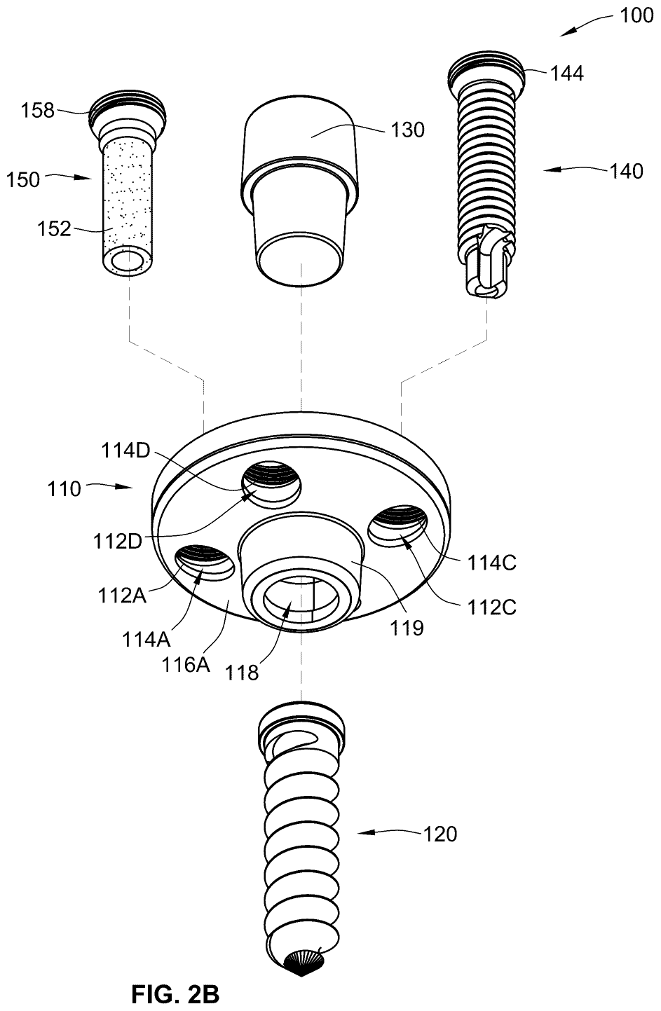

Referring generally to FIGS. 1A-2B, a modular prosthetic device 100 is shown according to some implementations of the present disclosure. The prosthetic device 100 includes a baseplate 110, a central screw 120 and a neck adapter extending in opposing directions from the center of the baseplate 110, a peripheral screw 140, and a peg 150. The peripheral screw 140 and the peg 150 are insertable into the baseplate 110.

The prosthetic device 100 includes a baseplate 110 with four peripheral bores 112A-D (which are best shown in FIGS. 2A and 2B) into which various fasteners may be inserted to secure the baseplate 110 to a bone. Although four peripheral bores 112A-D are shown in FIGS. 1A-2B, this is not required, and in some implementations, the baseplate 110 includes greater, or fewer, than four peripheral bores 112A-D. For example, the baseplate 110 can include one, two, three, five, six, or more peripheral bores 112A-D. Each of the peripheral bores 112A-D has threads 114A-D to cooperate with threaded heads of fasteners that are inserted into the peripheral bores 112A-D. However, this is not required, and in some implementations, one more of the peripheral bores 112A-D do not have threads 114A-D and fasteners are inserted, for example, via press-fit.

The baseplate 110 has a bone-contacting surface 116A (best shown in FIGS. 1B and 2B) and an opposing surface 116B (best shown in FIGS. 1A and 2A) that are on opposite surfaces of the baseplate 110. The bone-contacting surface 116A faces the bone in which the prosthetic device 100 is being inserted into. In some implementations, the bone-contacting surface 116A of the baseplate 110 has an osteogenic coating to promote bone ingrowth and/or outgrowth, which can aid in increasing fixation between the baseplate 110 and the bone. The baseplate 110 also includes a central bore 118 that extends through the center of the baseplate 110 and a boss 119. The boss 119 extends from the bone-contacting surface 116A of the baseplate 110. The boss 119 aids in positioning the baseplate 110 in bone of the patient. In some implementations, the boss 119 is made of the same material as the baseplate 110, and in further implementations, the boss 119 and the baseplate 110 are monolithic. In some implementations, the boss 119 has an osteogenic coating to promote bone ingrowth and/or ongrowth and further mitigate the potential of micromotion. However, this is not required, and in some implementations, the boss 119 does not have an osteogenic coating.

The prosthetic device 100 also includes a central screw 120 (which is best shown in FIGS. 2A and 2B) which inserts into and extends through the central bore 118 of the baseplate 110. In some implementations, the central screw 120 provides the main fixation of the prosthetic device 100. The central screw 120 also aids in compressing the bone that the central screw 120 is inserted into. Bone compression is advantageous to prosthetic device 100 installation because bone compression improves the stability of the prosthetic device 100 and facilitates osseointegration (bone ingrowth into the prosthetic device 100) and minimizes the potential of micromotion.

The central screw 120 also extends through the boss 119. The boss 119 provides additional thickness to the baseplate 110 to give the central screw 120 additional structure, which aids in, for example, preventing the baseplate 110 from rotating, and/or pivoting relative to the central screw 120.

The central screw 120 has a keyed top 122 to allow a tool, such as a screwdriver, to temporarily mate with the central screw 120 and facilitate the insertion of the central screw 120 into the central bore 118 (FIG. 2B) and into a bone. However, a keyed top 122 is not necessary, and in some implementations, the central screw 120 does not have a keyed top 122. In some such implementation, the central screw 120 can be inserted into the central bore 118 via a press fit, with the assistance of a tool such as a mallet and/or by any other suitable means. In some implementations, the central bore 118 is threaded and the central screw 120 has a threaded head to cooperate with each other. However, this is not required, and in some implementations, the central bore 118 is not threaded and/or the central screw 120 does not have a threaded head.

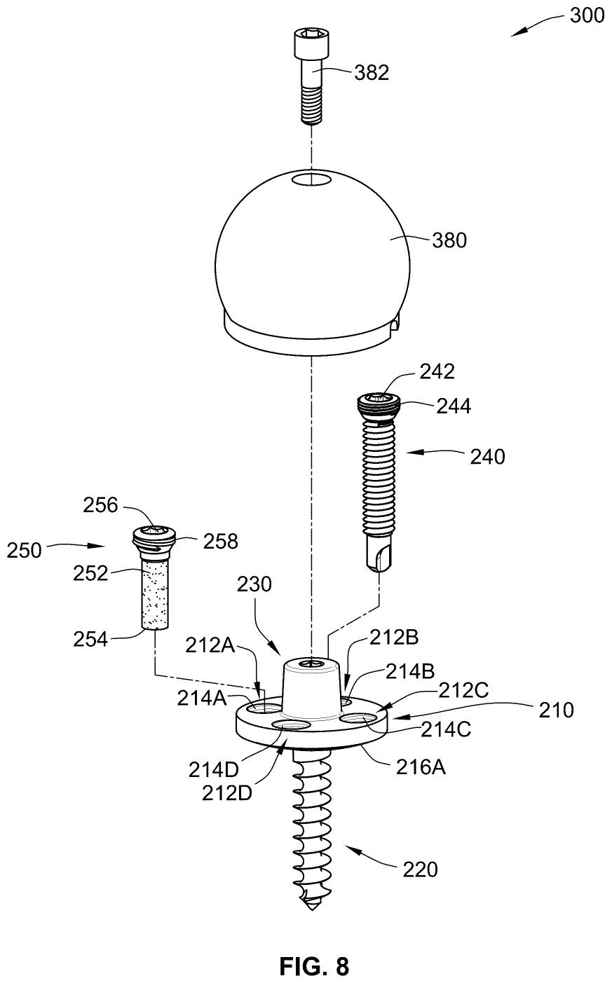

The prosthetic device 100 also includes a neck adapter 130 (best shown in FIGS. 2A and 2B) that is attachable to the baseplate 110. The neck adapter 130 can attach to the baseplate 110 via partial insertion into the central bore 118 (best shown in FIG. 2A). When coupled with the baseplate 110, the neck adapter 130 protrudes from the baseplate 110 in a direction opposite from the direction in which the central screw 120 extends. Although not shown in FIGS. 1A-2B, the neck adapter 130 is useful for attaching additional implant components, such as, for example a glenosphere (see for example glenosphere 380 in FIG. 8, which is described below). As shown, the central bore 118 is not threaded to aid in facilitating insertion of the neck adapter 130 into the central bore 118. However, in some alternative implementations, the neck adapter 130 can be threaded into the central bore 118.

The prosthetic device 100 also includes a peripheral screw 140. The peripheral screw 140 is insertable into one or more of the peripheral bores 112A-D. As depicted in FIGS. 1A-2B, the peripheral screw 140 is inserted into the right-most peripheral bore 112D. The peripheral screw 140 aids in stabilizing and securing the prosthetic device 100 into a bone. The peripheral screw 140, however, is not required, and in some implementations, the prosthetic device 100 does not have any peripheral screws 140. In other implementations, the prosthetic device 100 has one, two, three, or more peripheral screws 140.

The peripheral screw 140 has a keyed top 142 to allow a tool, such as a screwdriver, to temporarily mate with the peripheral screw 140 and facilitate the insertion of the peripheral screw 140 into the peripheral bore 112D (FIG. 2A) and into a bone. However, a keyed top 142 is not necessary, and in some implementations, the peripheral screw 140 does not have a keyed top 142. In some such implementations, the peripheral screw 140 can be inserted into the peripheral bore 112D via a press fit, with the assistance of a tool such as a mallet and/or by any other suitable means. The peripheral screw 140 also has a threaded head 144 that is able to cooperate with the threads 114D of the peripheral bore 112D that the peripheral screw 140 is inserted into. The threaded head 144 of the peripheral screw 140 aids in fixating the peripheral screw 140 into the peripheral bore 112D. However, the threaded head 144 of the peripheral screw 140 is not required, and in some implementations, the peripheral screw 140 does not have a threaded head 144. In such implementations, the peripheral screw 140 can be inserted into the peripheral bore 112D, for example, via a press fit, an adhesive, and/or any other suitable means.

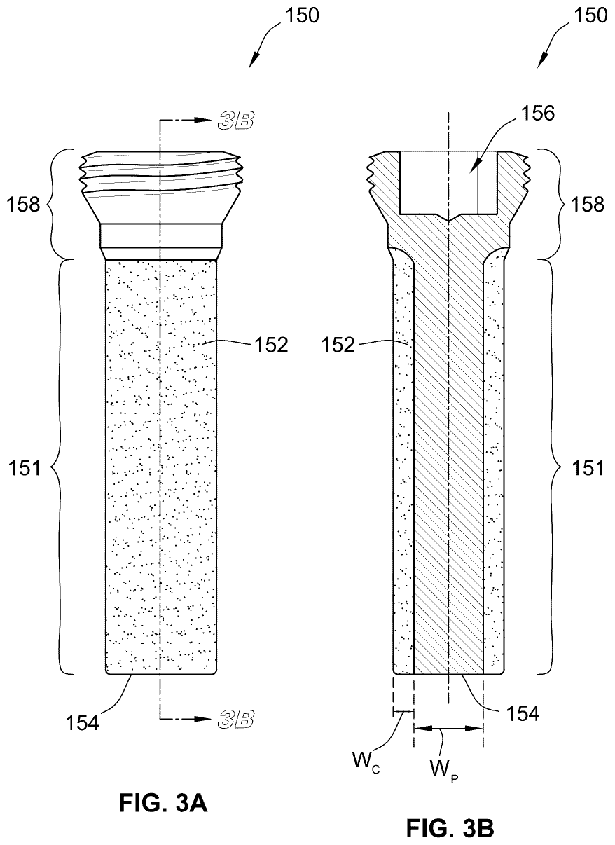

The prosthetic device 100 also includes a peg 150 that is configured to be inserted into one of the peripheral bores 112A-D and into a hole in a bone. The peg 150 has an osteogenic coating 152 (best shown in FIGS. 3A and 3B) that covers at least a portion of the surface of the peg 150 (as explained in more detail with respect to FIGS. 3A and 3B below) and helps promote bone ingrowth and/or outgrowth. The osteogenic coating 152 helps provide additional fixation of the peg 150 within the bone over time. The osteogenic coating 152, however, is not required, and in some implementations, the peg 150 does not have the osteogenic coating 152. There may be any suitable number of pegs 150 in the prosthetic device 100. For example, and in some implementations, there are none, one, two, three, four, five, or more pegs 150.

The peg 150 is designed having a length such that a distal end 154 of the peg 150 does not touch bone when the peg 150 is seated inside a hole in a bone. The distal end 154 of the peg 150 not touching bone is advantageous because a length of the peg 150 is not dictated by the depth of the prepared hole in a bone. Therefore, there is more flexibility in choosing a length of the peg 150. Additionally, the distal end 154 of the peg 150 not contacting bone prevents, or at least mitigates, bony ingrowth and/or outgrowth on the distal end 154 of the peg. The lack of, or mitigation of, bony ingrowth and/or ongrowth on the distal end 154 of the peg facilitates a subsequent revision, as compared to a prosthetic device 100 in which the distal end 154 of the peg 150 does contact bone. As such, in some implementations, the distal end 154 of the peg 150 lacks an osteogenic coating 152 to further mitigate bony ingrowth and/or outgrowth on the distal end 154 of the peg 150. However, this is not required, and in some implementations, the distal end 154 of the peg 150 touches bone and/or has an osteogenic coating 152, for example, to further increase fixation in the bone.

The distal end 154 of the peg 150 may be any of a variety of suitable shapes, including but not limited to: flat, conical, bumpy, grooved, rounded, etc., or any combination thereof. The peg 150 may also be any of a variety of suitable shapes, having a cross section with a circular shape, a triangular shape, a square shape, a conical shape, a tapered shape, a frustoconical shape, etc., or any combination thereof. One advantage of the peg 150 being at least partially tapered, at least near the distal end 154 of the peg 150, is that a tapered shape facilitates insertion of the peg 150 into a hole in a bone. Further, the peg 150 can be of any suitable length. In some implementations, the peg 150 has a length between about 10 millimeters and about 30 millimeters, between about 12 millimeters and about 28 millimeters, between about 15 millimeters and about 25 millimeters, between about 18 millimeters and about 22 millimeters, or any other suitable length.

The peg 150 also has a keyed top 156 to allow a tool, such as a screwdriver, to temporarily mate with the peg 150 and facilitate the insertion of the peg 150 into the peripheral bore 112A and into a hole in a bone. However, it should be noted that in some implementations, the peg 150 does not have a keyed top 156. In such implementations, for example, the peg 150 can be inserted into the peripheral bore 112A via a press fit, with the assistance of a tool such as a mallet and/or by any other suitable means

The peg 150 has a threaded head 158, for example, to cooperate with the threads 114A of the peripheral bore 112A that the peg 150 is inserted into. The threaded head 158 of the peg 150 aids in fixating the peg 150 into the peripheral bore 112A. However, the threaded head 158 of the peg 150 is not required, and in some implementations, the peg 150 does not have a threaded head 158. In such implementations, the peg 150 can be inserted into the peripheral bore 112A, for example, via a press fit, an adhesive, and/or any other suitable means.

The peg 150 and the peripheral screw 140 may have a variety of relationships in terms of their respective lengths. In some implementations, the peripheral screw 140 is longer than the peg 150. In other implementations, the peripheral screw 140 is shorter than, or of the same length as, the peg 150. For example, in some implementations, the length of the peripheral screw 140 is 5%-10% longer, 10%-15% longer, 15%-20% longer, 20%-25% longer, 25%-30% longer, 30%-40% longer, 40%-50% longer, 50%-60% longer, or any other percentage longer than the length of the peg 150.

The peg 150 and the peripheral screw 140 both aid in preventing rotational movement of the baseplate 110 relative to a central axis of the prosthetic device 100 (FIGS. 1A-1B). The peg 150 and the peripheral screw 140 also aid in providing additional fixation points for the prosthetic device 100 into bone. However, a greater number of pegs 150 and peripheral screws 140 increase the footprint of the prosthetic device 100 and therefore make removal of the prosthetic device 100 more difficult and potentially more damaging to the bone. Thus, the ideal number of peripheral screws 140 and/or pegs 150 of the prosthetic device 100 differs depending on the size, density, and type of bone in which the prosthetic device 100 is being installed. For example, larger and denser bones often can handle a larger number of peripheral screws 140 and/or pegs 150, whereas smaller and weaker bones may only be able to handle a single peripheral screw 140 and/or a single peg 150.

The peripheral screw 140 provides short-term and long-term stability to the prosthetic device 100. The peg 150 aids in providing enhanced long-term stability to the prosthetic device 100. In order to achieve enhanced long-term stability, the osteogenic coating 152 of the peg 150 promotes bone ingrowth and/or ongrowth along the sides of the peg 150. Thus, the peg 150 becomes relatively more fixated within bone over time as the bone ingrowth and/or ongrowth occurs within and to the osteogenic coating 152. While fixation of the sides of the peg 150 to bone is desirable for long-term stability, fixation of a distal end 154 of the peg 150 is avoided to facilitate a later removal of the peg 150, such as during a revision procedure.

During a revision procedure, the prosthetic device 100 is removed from the bone, which can cause stress to the bone supporting the prosthetic device 100, especially where there is bone ingrowth and/or ongrowth on prosthetic device 100. In order to mitigate the stress to the bone during a revision procedure, the distal end 154 of the peg 150 does not contact bone. Additionally, there is no osteogenic coating 152 on the distal end 154 of the peg 150 so that bone ingrowth and/or ongrowth is unlikely to occur on the distal end 154 of the peg 150. However, this is not required, and in some implementations, there is osteogenic coating 152 on the distal end 154 of the peg 150. In other implementations, the distal end 154 of the peg 150 has a coating that aids in preventing osteointegration. In some such implementations, the distal end portion of the peg 150 has a different shape than the cylindrical shape of the body 151 shown (e.g., a conical shape, a cone shape, spherical shape, etc., or any combination thereof). In some such implementations, the distal end of such a body is not coated with the osteogenic coating 152.

The ideal quantity of peripheral screws 140 and pegs 150 depends on the how much short-term and long-term stability, respectively, is desired, as well as the density, type, and size of the bone the prosthetic device 100 is being implanted in.

Referring to FIGS. 3A and 3B, additional details of the peg 150 are shown according to some implementations of the present disclosure. The peg 150 has a body 151 and a threaded head 158. As shown, the body 151 of the peg 150 lacks a thread. That is, the body 151 of the peg 150 is not threaded, which is at least one way the peg 150 is different from a screw-type fastener. When the peg 150 is coupled with, for example, the first peripheral bore 112A (as shown in FIG. 1A), the body 151 of the peg 150 extends from the bone-contacting surface 116A of the baseplate 110. As described above, the peg 150 has an osteogenic coating 152 to promote bony ingrowth and/or ongrowth. The osteogenic coating 152 can take any suitable form. For example, the osteogenic coating 152 can include one or more of fair or coarse particulate metal (such as titanium or alumina), hydroxyapatite, calcium phosphate, zoledronic acid, any other suitable material, or any combinations thereof. According to some implementations, the osteogenic coating 152 has a smooth surface, a bumpy surface, a grooved surface, or a combination thereof. In some implementations, a thickness w_{c} of the osteogenic coating 152 (FIG. 3B) can be uniform or nearly uniform about a length of the peg 150. However, such is not required. For example, in some implementations, the thickness, or width, w_{c} of the osteogenic coating 152 varies about a length of the peg 150. For example, according to some implementations, the osteogenic coating 152 has a uniform thickness, a non-uniform thickness, and/or a tapered thickness. In some implementations, the thickness w_{c} of the osteogenic coating 152 tapers along the length of the peg 150 towards the distal end 154 of the peg 150. In some such implementations, the tapered thickness w_{c} of the osteogenic coating 152 facilitates insertion of the peg 150 into a hole in a bone.

As can be seen best in FIG. 3B, a diameter of the peg 150 with the osteogenic coating 152 is greater than the diameter wₚ of a body 151 of the peg 150 alone. The diameter of the peg 150 with the osteogenic coating 152 is calculated as: (the diameter wₚ of the body 151 of the peg 150)+(2*(the thickness w_{c} of the osteogenic coating 152)). The diameter wₚ of the body 151 of the peg 150 and the width w_{c} of the osteogenic coating 152 can have any suitable measurement. The diameter wₚ of the body 151 of the peg 150 must be sufficient to be rigid and avoid breaking and the width w_{c} of the osteogenic coating 152 must be large enough to promote bony ingrowth/ongrowth.

In some implementations, the hole in the bone that the peg 150 is inserted into has a diameter that is greater than or equal to the diameter of the body 151 of the peg 150 alone, but the diameter of the hole is also smaller than the diameter of the peg 150 (inclusive of the body 151 of the peg 150 and the osteogenic coating 152). In such implementations, when the peg 150 is inserted into the hole, the peg 150 compacts the bone due to the outwardly extending force of the osteogenic coating 152 against the bone. This compaction of the bone assists in securing the peg 150 within the bone and promotes bone ingrowth and/or ongrowth on the outer surface of the peg 150.

The thickness w_{c} of the osteogenic coating 152 may be defined relative to the diameter wₚ of the peg 150. For example, in some implementations, the ratio of the thickness w_{c} of the osteogenic coating 152: the diameter wₚ of the body 151 of the peg 150 is in a range of about 1:50 to about 1:1, of about 1:25 to about 1:2, and/or of about 1:10 to 1:3. The thickness w_{c} of the osteogenic coating 152 is in a range from about 0.1 mm to about 1.5 mm, from about 0.2 to about 1 mm, from about 0.3 to about 0.8 mm, and/or from about 0.4 to about 0.6 mm. In some implementations, the thickness w_{c} of the osteogenic coating 152 is about 0.1 mm, about 0.15 mm, about 0.2 mm, about 0.25 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, or any number in between. In some implementations, the diameter wₚ of the body 151 of the peg 150 is in a range from about 1 mm to about 5 mm, from about 1.5 mm to about 4 mm, and/or from about 2 mm to about 3 mm. In some implementations, the diameter wₚ of the body 151 of the peg 150 is about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, or any number in between.

The osteogenic coating 152 may be applied in various ways. For example, the osteogenic coating 152 may be applied as a single layer or in multiple layers. Further, the osteogenic coating 152 may be electroplated, spray coated, and/or 3D printed in a single pass or in multiple passes. The osteogenic coating 152 may be applied to the distal end 154 of the peg 150 to be later removed, or the osteogenic coating 152 may never be applied to the distal end 154. However, this is not required, and in some implementations, the osteogenic coating 152 remains on the distal end 154 of the peg 150.

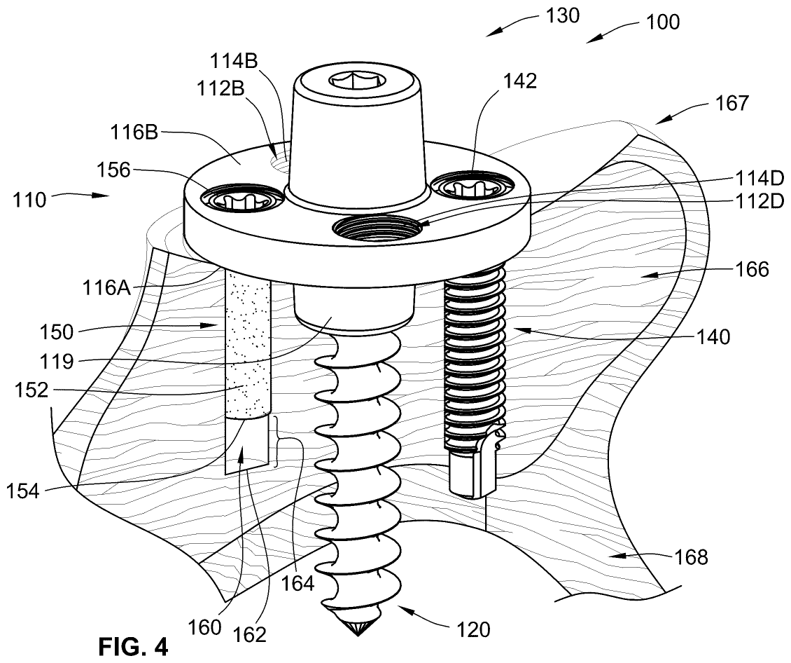

While the discussion of FIGS. 1A-3B describes a prosthetic device 100 including a peg 150, the discussion of FIG. 4 provides additional detail regarding use of such a prosthetic device 100 in bone of a patient.

Referring to FIG. 4, a cross-sectional view of the prosthetic device 100 implanted in a shoulder 167 of a patient is shown. Although shown in a shoulder, the prosthetic device 100 may be used in any appropriate location. For example, the prosthetic device 100 is usable in a knee, hip, foot, ankle, etc.

The central screw 120 of the prosthetic device may be inserted into the central bore 118 and extend through the boss 119 and through a hole in a bone. The peg 150 is inserted into one of the peripheral bores 112A and extends through a peripheral hole in the bone 160. A peripheral screw 140 is also inserted into one of the peripheral bores 112C and through a hole in a bone. The peripheral screw 140 is bi-cortical because the peripheral screw 140 extends through a cortical portion of bone 168, through a cancellous portion of bone 166, and then again through the cortical portion of bone 168. Because cortical bone 168 is stronger than cancellous bone 166, the peripheral screw 140 is able to achieve bi-cortical fixation which provides good short-term stability of the prosthetic device 100. Bi-cortical fixation of the peripheral screw 150, however, is not always possible. For example, for smaller and/or weaker bones, a peripheral screw 150 may not be able to be bi-cortically fixated without causing excessive harm to the bone. Thus, in some implementations, especially where bi-cortical fixation of the peripheral screw 150 is not medically recommended, the prosthetic device 100 does not use any peripheral screws 140. In some such implementations, only the peg 150, or multiple pegs 150, are inserted into the peripheral bores 112A-D. The peg 150 is able to achieve additional stability of the prosthetic device 100 over time as bone ingrowth and/or ongrowth occurs in the osteogenic coating 152 of the peg 150.

As shown in FIG. 4, the hole 160 in the bone that the peg 150 is inserted into has a bottom 162. The distal end 154 of the peg 150 does not contact the bottom 162 of the hole 160 in the bone, nor does the distal end 154 of the peg 150 generally touch bone. The distal end 154 of the peg 150 does not have significant contact with bone in order to facilitate a later removal of the peg 150 from the hole 160 in the bone. For example, by having no contact between the distal end 154 of the peg 150 and bone, there is no direct force on the bone at the bottom 162 of the hole during removal of the peg 150. To further prevent any direct force on the bone at the bottom 162 of the hole 160 during removal of the peg 150, in some implementations, the distal end 154 of the peg 150 does not have the osteogenic coating 152. Bone ingrowth and/or ongrowth on the distal end 154 of the peg 150 is therefore not promoted by the osteogenic coating 152. The lack of osteogenic coating 152 on the distal end 154 of the peg 150 is advantageous for a later removal of the peg 150 because bone cannot easily grow and/or attach to the distal end 154 of the peg 150.

To avoid contact between the distal end 154 of the peg 150 and bone, a depth of the hole 160 in the bone (distance between the bone-contacting surface 116A of the baseplate 110 and the bottom 162 of the hole 160) is greater than the length of the peg 150 inserted into the hole 160 (distance between the bone-contacting surface 116A of the baseplate 110 and the distal end 154 of the peg 150). Because the length of the hole 160 in the bone is greater than the length of the peg 150, the distal end 154 of the peg 150 is unable to reach or contact the bottom 162 of the hole 160, allowing for greater flexibility in choosing a length of the peg 150. For example, in some implementations, the depth of the hole 160 is 5%-10% longer, 10%-15% longer, 15%-20% longer, 20%-25% longer, 25%-30% longer, 30%-40% longer, 40%-50% longer, 50%-60% longer, or any other percentage longer than the length of the peg 150. In some implementations, the length of the peg 150 (which is measured from the distal end 154 of the peg 150 to the bone-contacting surface 116A of the baseplate 110, when the peg 150 is fully seated in the baseplate 110) is between about 10 millimeters and about 30 millimeters. In some implementations, the length of the hole 160 (which is measured from the bottom 162 of the hole 160 to the bone-contacting surface 116A of the baseplate 110) is between about 11 millimeters and about 34 millimeters. In some implementations the distal end 154 of the peg 150 is at least about 1 millimeter from the bottom 162 of the hole 160 when the peg 150 is fully seated in the baseplate 110. In some implementations, the distal end 154 of the peg 150 is at least about 2, 3, 4, 5, 6, 7, 8, 9, 10 millimeters from the bottom 162 of the hole 160 when the peg 150 is fully seated in the baseplate 110. The spacing between the distal end 154 of the peg 150 and the bottom 162 of the hole 160 aids in avoiding osteointegration of the distal end 154 of the peg 150 with the bone.

In some implementations, the length of the hole in the bone 160 and the length of the peg 150 are selected based on the location of cancellous bone 166 and/or cortical bone 168. For example, as shown in FIG. 4, the length of the hole in the bone 160 is such that the bottom 162 of the hole 160 is in cortical bone 168 and the length of the peg 150 is such that the distal end 154 of the peg 150 is in cancellous bone 166. Because cancellous bone 166 is more easily compacted than cortical bone 168, the insertion of the peg 150 into cancellous bone 166 causes less stress to the bone than insertion of the peg 150 into cortical bone 168. Having the length of the hole 160 in the bone such that the bottom 162 of the hole 160 is in cortical bone 168 also presents an advantage. In such implementations, the distal end 154 of the peg 150 does not contact cortical bone 168. Because the distal end 154 of the peg 150 does not contact cortical bone 168, a geometry of the peg 150 does not need to be capable of cutting through cortical bone 168. Therefore, a shaft of the peg 150 may be fully covered in the osteogenic coating 152 to maximize bony ingrowth/ongrowth on the peg 150.

Because the distal end 154 of the peg 150 does not contact the bottom 162 of the hole 160, there is a gap 164 between the distal end 154 of the peg 150 and the bottom 162 of the hole 160 into which the peg 150 is inserted. In some implementations, the distance of this gap 164 is in a range of about 0.5 mm to about 30 mm, of about 1 mm to about 20 mm, of about 2 mm to about 10 mm, of about 3 mm to about 5 mm, of about 3.5 mm to about 4.5 mm, and/or about 4 mm.

In accordance with some implementations, the hole 160 in the bone has a diameter greater than or equal to the diameter wₚ of the peg 150 itself. In some implementations, the thickness of the osteogenic coating w_{c} causes the diameter of the peg 150 with the osteogenic coating 152 to have a total diameter (2*w_{c}+wₚ) that exceeds the diameter of the hole 160 in the bone. In such implementations, when the peg 150 is inserted into the hole 160 in the bone, the bone is compacted by the osteogenic coating 152. This compaction assists in securing the peg 150 within the bone and promoting bone ingrowth and/or ongrowth on the outer surface of the peg 150.

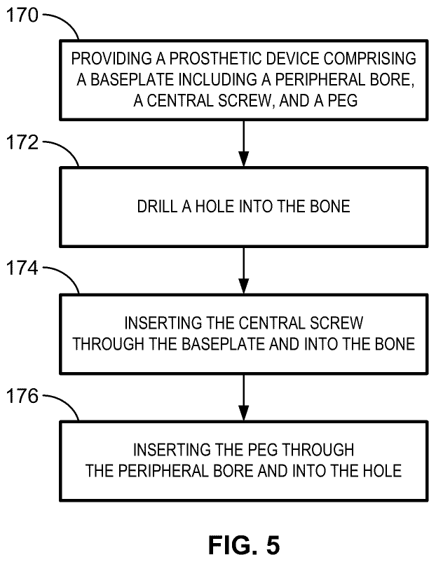

Referring to FIG. 5, a method of installing a prosthetic device into bone is described according to some implementations of the present disclosure. The method begins with step 170 by providing a prosthetic device. In some implementations, the prosthetic device comprises a baseplate including a peripheral bore, a central screw, and a peg. The prosthetic device may be any suitable prosthetic device such as, for example the prosthetic devices 100, 200 described herein.

The method continues with step 172 where a hole is drilled into a bone. For example, a surgeon can drill the hole into the bone using one or more bill bits. The bone into which the hole is drilled can be any suitable bone. For example, the bone may be a humeral and/or scapula bone in the circumstance of a shoulder joint replacement, or a tibial and/or femoral bone in the circumstance of a knee joint replacement. Drilling may be accomplished with the help of a tool. For example, a drill, a hammer, a drill bit, pin vise, Dremel, drill press, or any other suitable tool may be used. The hole can be of any suitable diameter and length, as discussed in more detail herein.

The method continues with step 174 where the central screw is inserted through the baseplate and into the bone. The central screw aids in providing fixation for the baseplate against the bone. The central screw may be inserted into a hole in the bone or may create its own path through the bone while being screwed into the bone (e.g., is a self-tapping screw).

The method continues with step 176 where a peg is inserted through the peripheral bore and into the hole that was drilled. The peg aids in providing fixation for the baseplate against the bone and aids in preventing the baseplate from rotating or pivoting relative to the central screw. In some implementations of the method, the length of the peg is smaller than the length of the hole so that a distal end of the peg does not touch bone. No contact between the distal end of the peg and bone is desirable for allowing greater flexibility in choosing a length of the peg and mitigating stress to the bone during a subsequent revision and/or removal of the prosthetic device.

In some implementations, the operations do not necessarily occur in the order depicted in the flow diagram of FIG. 5. As one example, step 176 can occur before step 174. For example, the peg may be monolithic with the baseplate, or the peg may be inserted into the peripheral bore and into the hole prior to the central screw being inserted through the baseplate and into the hole. Having step 176 occur before step 174 can be useful for providing additional rotational stability to the prosthetic device when inserting the central screw through the baseplate and into the bone.

Additionally, in some implementations, the operations depicted in FIG. 5 may occur in series and/or in parallel. As one example, step 170 may occur simultaneously, or at least partially simultaneously, with steps 172, 174, and/or 176. As further examples, the step 172 may occur simultaneously, or at least partially simultaneously, with steps 170, 174, and/or 176; step 174 may occur simultaneously, or at least partially simultaneously, with steps 170, 172, and/or 176; and/or step 176 may occur simultaneously, or at least partially simultaneously, with steps 170, 172, and/or 174.

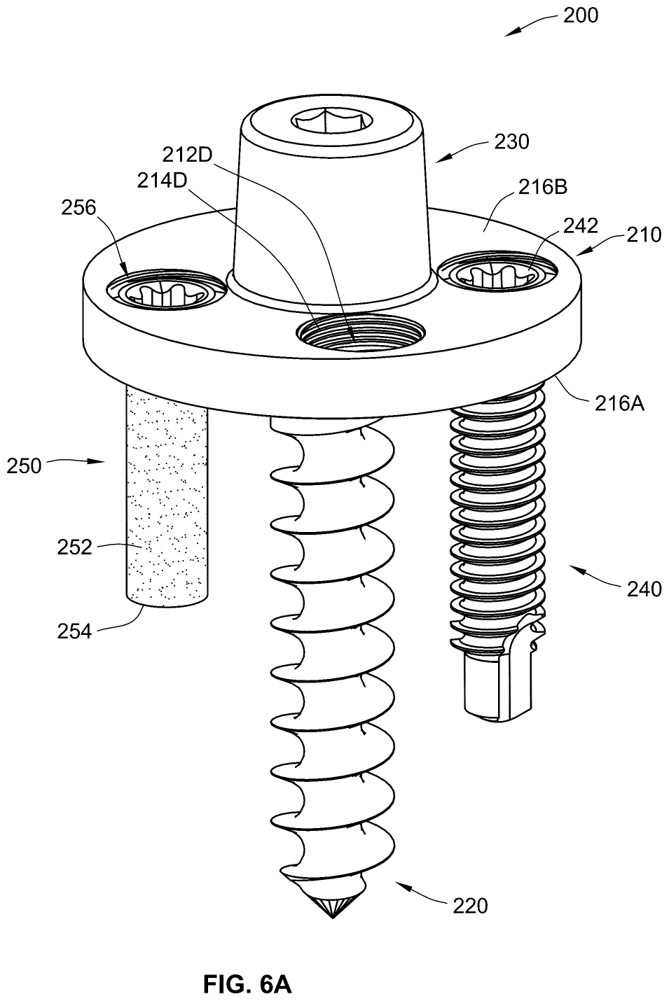

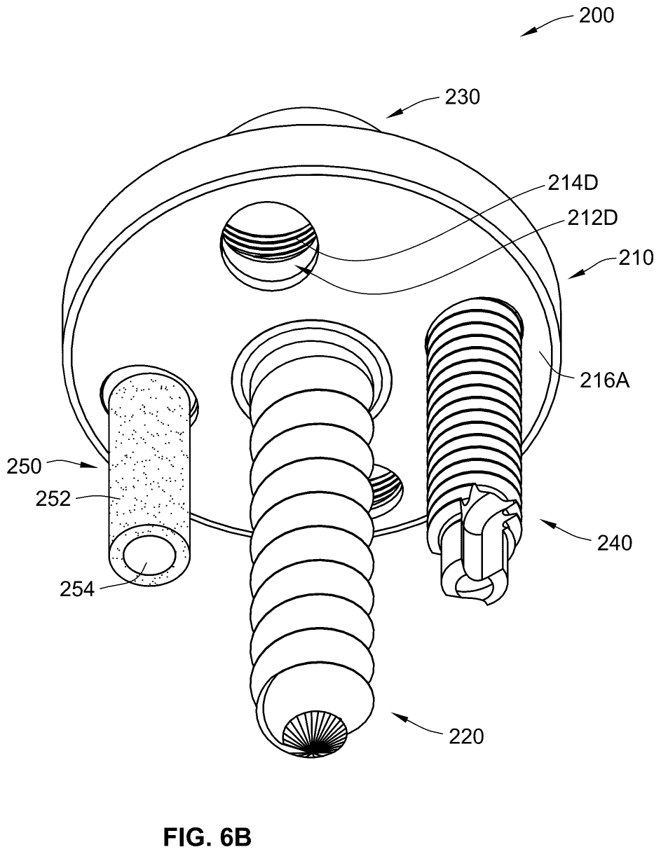

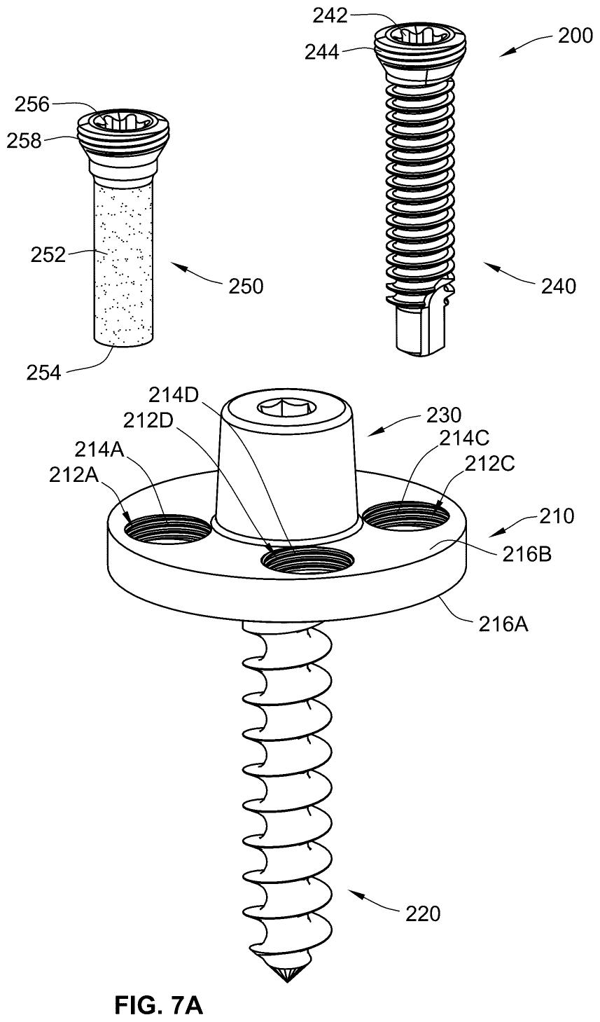

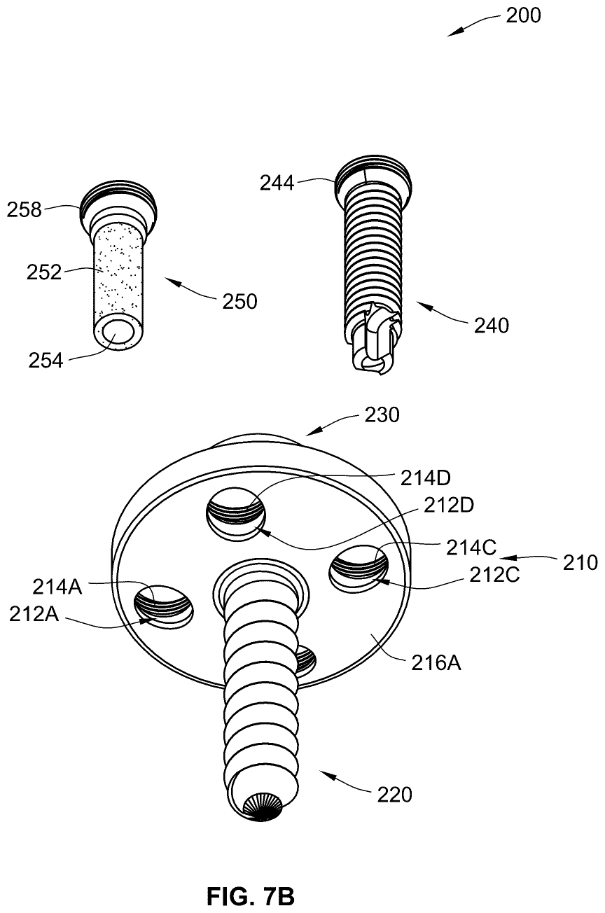

While the discussion of FIGS. 1A-5 describe a prosthetic device 100 including a peg 150 and a central screw 120, the discussion of FIGS. 6A-7B provide additional details regarding a prosthetic device 200 including a peg 250 in which the central screw 220 is monolithically formed with a baseplate 210.

Referring to FIGS. 6A-7B, a monolithic prosthetic device 200 is shown according to some implementations of the present disclosure. The monolithic prosthetic device 200 includes a baseplate 210, a central screw 220, a neck adapter 230, a peripheral screw 240, and a peg 250, which are the same as, or similar to, the baseplate 110, the central screw 120, the neck adapter 130, the peripheral screw 140, and the peg 150, except that the monolithic prosthetic device 200 differs in that the baseplate 210, the central screw 220, and the neck adapter 230 are monolithic and not separate and distinct elements like the baseplate 110, the central screw 120, and the neck adapter 130 of the prosthetic device 100 described herein.

An advantage of the central screw 220 being monolithic with the baseplate 210 is that rotational and/or pivotal movement of the baseplate 210 relative to the central screw 220 is avoided. Additionally, the central screw 220 being monolithic with the baseplate 210 mitigates the marginal utility of using a boss to provide additional stability to the relationship between the central screw 220 and the baseplate 210. The baseplate 210 not including a boss aids in reducing the footprint of the baseplate 210 on the bone into which the prosthetic device 200 is installed. However, according to some implementations, the monolithic prosthetic device 200 does include a boss connected to the baseplate 210 and through which the central screw 220 extends. In such implementations, the boss assists in reinforcing the monolithic connection between the central screw 220 and the baseplate 210.

FIG. 8 is a top perspective exploded view of a monolithic prosthetic device 300 that is the same as the monolithic prosthetic device 200 of FIGS. 6A-7B, but differs in that the monolithic prosthetic device 300 includes an implant component 380, according to some implementations.

The neck adapter 230 cooperates with the implant component 380. As shown in FIG. 8, the implant component 380 is a glenosphere. However, the implant component 380 does not need to be a glenosphere. For example, the implant component 380 can be a socket, a liner, a spacer, a femoral component, a tibial component, and/or any other suitable component of an implant. The implant component 380 is fixated to the prosthetic device 200 via an implant component screw 382. In some implementations, the implant component screw 382 extends through the implant component 380 and into the neck adapter 230. An implant component screw 382, however, is not required. Instead, and in some implementations, the implant component 380 is attached to the prosthetic device 200 via an adhesive, nails, sutures, etc., or a combination thereof. While the implant component 380 and implant component screw 382 are shown with the monolithic prosthetic device 200, this is not required. Accordingly, in some implementations, the implant component 380 and implant component screw 382 are used with the modular prosthetic device 100 described herein.

In summary, the implementations discussed above present a number of advantages over current and past prosthetic devices and practices. Because the peg does not need to be as long as the peripheral screws, the prosthetic device is a viable option for more types, sizes, and conditions of bone than prosthetic devices that don't utilize a peg. Additionally, the osteogenic coating of the peg provides long-term stability of the prosthetic device, increasing the overall construct fixation.

1. A prosthetic device comprising:
a baseplate having a bone-contacting surface and a peripheral bore;
a central screw extending from the bone-contacting surface of the baseplate; and
a peg coupled to the peripheral bore of the baseplate such that the peg is configured to extend a first length beyond the bone-contacting surface of the baseplate and into a hole formed in a bone, the hole having a first diameter and a depth that is greater than the first length such that a distal end of the peg and a bottom of the hole define a gap, wherein sides of the peg include an osteogenic coating such that the peg, inclusive of the osteogenic coating, has a second diameter that is greater than the first diameter of the hole, and wherein the distal end of the peg does not include the osteogenic coating.

2. The prosthetic device of claim 1, wherein the osteogenic coating comprises particulate metal.

3. (canceled)

4. The prosthetic device of claim 1, wherein a body of the peg has a third diameter that is less than the first diameter of the hole such that the osteogenic coating, and not the body of the peg, interferes with the hole in the bone to aid in osseointegration.

5. The prosthetic device of claim 1, wherein the bone-contacting surface of the baseplate includes an osteogenic coating.

6. The prosthetic device of claim 1, wherein the peg extends into the bone such that a distal end of the peg is in a cancellous portion of the bone.

7. The prosthetic device of claim 1, further comprising a peripheral screw configured to be coupled to a second peripheral bore of the baseplate such that the peripheral screw extends a second length beyond the bone-contacting surface, the second length being greater than the first length.

8. (canceled)

9. The prosthetic device of claim 1, wherein the gap is at least one millimeter.

10. (canceled)

11. The prosthetic device of claim 1, wherein the peripheral bore includes a threaded portion.

12-17. (canceled)

18. The prosthetic device of claim 1, further including a neck adapter configured to be coupled to the baseplate such that the neck adapter protrudes from a surface of the baseplate that opposes the bone-contacting surface of the baseplate.

19. The prosthetic device of claim 18, wherein the baseplate further includes a central bore.

20. The prosthetic device of claim 19, wherein the neck adapter is configured to be at least partially insertable into the central bore of the baseplate.

21. The prosthetic device of claim 1, further including a boss extending from the bone-contacting surface of the baseplate.

22. The prosthetic device of claim 21, wherein the boss and the baseplate are monolithic.

23. The prosthetic device of claim 21, wherein the baseplate further includes a central bore, and the central screw is configured to be at least partially insertable through the central bore and at least partially through the boss.

24. The prosthetic device of claim 1, wherein the peg and the central screw are generally parallel to each other.

25. The prosthetic device of claim 1, wherein the peg is generally perpendicular to the baseplate.

26. The prosthetic device of claim 1, wherein the peg is shorter length-wise than the central screw.
